SEQUENCE LISTING (26 sequences)

SEQ ID NO: 1 - protein, 299 aa, Homo sapiens [sequence omitted]

SEQ ID NO: 2 - protein, 128 aa, Mus sp.
MDLSELERDNTGRCRLSSPVPAVCLKEPCVLGVDEAGRGPVLGPMVYAICYCPLSRLADLEALKVADSKTLTENERERLFAKMEEDGDFVGWALDVLSPNLISTSMLGRVKYNLNSLSHDTAAGLIQY

SEQ ID NO: 3 - protein, 307 aa, Caenorhabditis elegans
SKTVKYFIERMSLKCETERSKTWNNFGNGIPCVLGIDEAGRGPVLGPMVYAAAISPLDQNVELKNLGVDDSKALNEAKREEIFNKMNEDEDIQQIIAYALRCLSPELISCSMLKRQKYSLNEVSHEAAITLIRDALACNVNVVEIKVDTVGPKATYQAKLEKLFPGISICVTEKADSLFPIVSAASIAAKVTRDSRLRNWQFREKNIKVPDAGYGSGYPGDPNTKKFLQLSVEPVFGFCSLVRSSWKTASTIVEKRCVPGSWEDDEEEGKSQSKRMTSWMVPKNETEVVPKRNMEINLTKIVSTLFL

SEQ ID NO: 4 - protein, 307 aa, Saccharomyces cerevisiae
MVPPTVEASLESPYTKSYFSPVPSALLEQNDSPIIMGIDEAGRGPVLGPMVYAVAYSTQKYQDETIIPNYEFDDSKKLTDPIRRMLFSKIYQDNEELTQIGYATTCITPLDISRGMSKFPPTRNYNLNEQAHDVTMALIDGVIKQNVKLSHVYVDTVGPPASYQKKLEQRFPGVKFTVAKKADSLYCMVSVASVVAKVTRDILVESLKRDPDEILGSGYPSDPKTVAWLKRNQTSLMGWPANMVRFSWQTCQTLLDDASKNSIPIKWEEQYMDSRKNAAQKTKQLQLQMVAKPVRRKRLRTLDNWYR

SEQ ID NO: 5 - protein, 198 aa, Escherichia coli
MIEFVYPHTQLVAGVDEVGRGPLVGAVVTAAVILDPARPIAGLNDSKKLSEKRRLALYEEIKEKALSWSLGRAEPHEIDELNILHATMLAMQRAVAGLHIAPEYVLIDGNRCPKLPMPAMAVVKGDSRVPEISAASILAKVTRDAEMAALDIVFPQYGFAQHKGYPTAFHLEKLAEHGATEHHRRSFGPVKRALGLAS

SEQ ID NO: 6 - DNA, 17 bp, Artificial
  Antisense oligonucleotide
ccacaccgacggcgccc

SEQ ID NO: 7 - DNA, 15 bp, Artificial
  Antisense oligonucleotide
cacaccgacggcgcc

SEQ ID NO: 8 - DNA, 13 bp, Artificial
  Antisense oligonucleotide
acaccgacggcgc

SEQ ID NO: 9 - DNA, 11 bp, Artificial
  Antisense oligonucleotide
caccgacggcg

SEQ ID NO: 10 - DNA, 26 bp, Artificial
  Sense primer
acgctggccgggagtcgaaatgcttc

SEQ ID NO: 11 - DNA, 28 bp, Artificial
  Sense primer
ctgttcctggcccacagagtcgccttgg

SEQ ID NO: 12 - DNA, 29 bp, Artificial
  Sense primer
ggtctttctgacctggaatgagtgcagag

SEQ ID NO: 13 - DNA, 29 bp, Artificial
  Antisense primer
cttgcctggtttcgccctccgattcttgt

SEQ ID NO: 14 - DNA, 29 bp, Artificial
  Antisense primer
ttgattttcatgcccttctgaaacttccg

SEQ ID NO: 15 - DNA, 34 bp, Artificial
  Antisense primer
cctcatcctctatggcaaacttcttaaatctggc

SEQ ID NO: 16 - DNA, 20 bp, Artificial
  Sense primer
agcaggcgccgcttcgaggc

SEQ ID NO: 17 - DNA, 26 bp, Artificial
  Sense primer
cccgctcctgcagtattagttcttgc

SEQ ID NO: 18 - DNA, 25 bp, Artificial
  Sense primer
ttgcagctggtggtggcggctgagg

SEQ ID NO: 19 - DNA, 26 bp, Artificial
  Antisense primer
tccaatagggtctttgagtctgccac

SEQ ID NO: 20 - DNA, 25 bp, Artificial
  Antisense primer
cactttcagcgcctccagatctgcc

SEQ ID NO: 21 - DNA, 26 bp, Artificial
  Antisense primer
gcgaggcaggggacaataacagatgg

SEQ ID NO: 22 - DNA, 1131 bp, Homo sapiens
cgcgcctgcagtattagttcttgcagctggtggtggcggctgaggcggcatggatctcagcgagctggagagagacaatacaggccgctgtcgcctgagttcgcctgtgcccgcggtgtgccgcaaggagccttgcgtcctgggcgtcgatgaggcgggcaggggccccgtgctgggccccatggtctacgccatctgttattgtcccctgcctcgcctggcagatctggaggcgctgaaagtggcagactcaaagaccctattggagagcgagcgggaaaggctgtttgcgaaaatggaggacacggactttgtcggctgggcgctggatgtgctgtctccaaacctcatctctaccagcatgcttgggtgggtcaaatacaacctgaactccctgtcacatgatacagccactgggcttatacagtatgcattggaccagggcgtgaacgtcacccaggtattcgtggacaccgtagggatgccagagacataccaggcgcggctgcagcaaagttttcccgggattgaggtgacggtcaaggccaaagcagatgccctctacccggtggttagtgctgccagcatctgtgccaaggtggcccgggaccaggccgtgaagaaatggcaattcgtggagaaactgcaggacttggatactgattatggctcaggctaccccaatgatcccaagacaaaagcgtggttgaaggagcacgtggagcctgtgttcggcttcccccagtttgtccggttcagctggcgcacggcccagaccatcctggagaaagaggcggaagatgttatatgggaggactcagcatccgagaatcaggagggactcaggaagatcacatcctacttcctcaatgaagggtcccaagcccgtccccgttcttcccaccgatatttcctggaacgcggcctggagtcagcaaccagcctctagcagctgcctctacgcgctctacctgcttccccaacccagacattaaaattgtttaaggagaaccacacgtaggggatgtacttttgggacagaagcaaggtgggagtgtgctctgcagccgggtccagctacttccttttggaaccttaaatagaatgggtgttggttgataaaaaaaaaaa

SEQ ID NO: 23 - DNA, 17 bp, Artificial
  Synthetic Ras RNA fragment for use in RNase H cleavage assay
gggcgccgtcggtgtgg

SEQ ID NO: 24 - DNA, 20 bp, Homo sapiens
agcttctttgcacatgtaaa

SEQ ID NO: 25 - DNA, 20 bp, Mus sp.
tccagcactttcttttccgg

SEQ ID NO: 26 - DNA, 20 bp, Mus sp.
ccggtaccccaggttcttca

(19) United States
(12) Patent Application Publication
(10) Pub. No.: US 2001/0044145 A1
(43) Pub. Date: Nov. 22, 2001

(54) Methods of using mammalian RNase H and compositions thereof

(76) Inventors: Brett P. Monia, Encinitas, CA (US); Phillip Dan Cook, Fallbrook, CA (US); Stanley T. Crooke, Carlsbad, CA (US); Walter Lima, San Diego, CA (US); Hongjiang Wu, Carlsbad, CA (US)

Correspondence Address: Jane Massey Licata, Licata & Tyrrell P.C., 66 East Main Street, Marlton, NJ, 08053, US

(21) Appl. No.: 09/799,848

(22) Filed: Mar. 5, 2001

Related U.S. Application Data
(63) Continuation-in-part of application No. 09/781,712, filed on Feb. 12, 2001.
Application No. 09/781,712 is a continuation-in-part of application No. 09/684,254, filed on Oct. 6, 2000.
Application No. 09/684,254 is a continuation of application No. 09/343,809, filed on Jun. 30, 1999, now abandoned.
Application No. 09/343,809 is a continuation of application No. 09/203,716, filed on Dec. 2, 1998, now Pat. No. 6,001,653.
(63) Continuation-in-part of application No. 09/453,514, filed on Dec. 1, 1999.
Application No. 09/453,514 is a division of application No. 09/144,611, filed on Aug. 31, 1998, now Pat. No. 6,146,829.
Application No. 09/144,611 is a division of application No. 08/861,306, filed on Apr. 21, 1997, now Pat. No. 5,856,455.
Application No. 08/861,306 is a division of application No. 08/244,993, filed on Jun. 21, 1994, now Pat. No. 5,623,065.
Application No. 08/244,993 is a continuation-in-part of application No. 07/814,961, filed on Dec. 24, 1991, now abandoned.
(63) Continuation-in-part of application No. 09/462,280, filed on Mar. 1, 2000.
Application No. 09/462,280 is a 371 of international application No. PCT/US98/13966, filed on Jul. 6, 1998.
(60) Provisional application No. 60/067,458, filed on Dec. 4, 1997.

(51) Int. Cl. A61K 48/00; C12N 9/22

(52) U.S. Cl. 435/199; 514/44

(57) ABSTRACT

The present invention relates to methods for using mammalian RNase H and compositions thereof, particularly for reduction of a selected cellular RNA target via antisense technology.

This application is a continuation-in-part of U.S. patent application Ser. No. 09/781,712, filed Feb. 12, 2001, which is a continuation-in-part of U.S. patent application Ser. No. 09/684,254, filed Oct. 6, 2000, which is a continuation of U.S. patent application Ser. No. 09/343,809, filed Jun. 30, 1999, which is a continuation of U.S. patent application Ser. No. 09/203,716, filed Dec. 2, 1998, now issued as U.S. Pat. No. 6,001,653, which claimed the benefit of priority from U.S. Provisional Application 60/067,458, filed Dec. 4, 1997, now abandoned.

This application is also a continuation-in-part of U.S. patent application Ser. No. 09/453,514, filed Dec. 1, 1999, which is a divisional of U.S. patent application Ser. No. 09/144,611, filed Aug. 31, 1998, now issued as U.S. Pat. No. 6,146,829. U.S. Ser. No. 09/144,611 is a divisional of U.S. patent application Ser. No. 08/861,306, filed Apr. 21, 1997, now issued as U.S. Pat. No. 5,856,455, which itself is a divisional of U.S. patent application Ser. No. 08/244,993, filed on Jun. 21, 1994, now issued as U.S. Pat. No. 5,623,065. U.S. Ser. No. 08/244,993 is a continuation-in-part of U.S. patent application Ser. No. 07/814,961, filed Dec. 24, 1991, now abandoned.

This application is also a continuation-in-part of U.S. patent application Ser. No. 09/462,280, filed Mar. 1, 2000, which was the National Stage of International Application No. PCT/US98/13966, filed Jul. 6, 1998, which is a foreign filing of U.S. patent application Ser. No. 08/889,296, filed Jul. 8, 1997, now issued as U.S. Pat. No. 5,872,242, which is a continuation-in-part of U.S. patent application Ser. No. 08/411,734, filed Apr. 3, 1995, which in turn is a continuation-in-part of U.S. patent application Ser. No. 08/007,996, filed Jan. 21, 1993, now abandoned.

Each of the above-referenced patent applications is incorporated herein in its entirety.

### FIELD OF THE INVENTION

The present invention relates to methods for using mammalian RNase H and compositions thereof, particularly for reduction of selected cellular RNA via antisense technology.

### BACKGROUND OF THE INVENTION

RNase H hydrolyzes RNA in RNA-DNA hybrids. This enzymatic activity was first identified in calf thymus but has subsequently been described in a variety of organisms (Stein, H. and Hausen, P., Science, 1969, 166, 393-395; Hausen, P. and Stein, H., Eur. J. Biochem., 1970, 14, 278-283). RNase H activity appears to be ubiquitous in eukaryotes and bacteria (Itaya, M. and Kondo K. Nucleic Acids Res., 1991, 19, 4443-4449; Itaya et al., Mol. Gen. Genet., 1991 227, 438-445; Kanaya, S., and Itaya, M., J. Biol. Chem., 1992, 267, 10184-10192; Busen, W., J. Biol. Chem., 1980, 255, 9434-9443; Rong, Y. W. and Carl, P. L., 1990, Biochemistry 29, 383-389; Eder et al., Biochimie, 1993 75, 123-126). Although RNases H constitute a family of proteins of varying molecular weight, nucleolytic activity and substrate requirements appear to be similar for the various isotypes. For example, all RNases H studied to date function as endonucleases, exhibiting limited sequence specificity and requiring divalent cations (e.g., Mg²⁺, Mn²⁺) to produce cleavage products with 5′ phosphate and 3′ hydroxyl termini (Crouch, R. J., and Dirksen, M. L., Nuclease, Linn, S, M., & Roberts, R. J., Eds., Cold Spring Harbor Laboratory Press, Plainview, N.Y. 1982, 211-241).

RNase HI from E. coli is the best-characterized member of the RNase H family. The 3-dimensional structure of E. coli RNase HI has been determined by x-ray crystallography, and the key amino acids involved in binding and catalysis have been identified by site-directed mutagenesis (Nakamura et al., Proc. Natl. Acad. Sci. USA, 1991, 88, 11535-11539; Katayanagi et al., Nature, 1990, 347, 306-309; Yang et al., Science, 1990, 249, 1398-1405; Kanaya et al., J. Biol. Chem., 1991, 266, 11621-11627). The enzyme has two distinct structural domains. The major domain consists of four α helices and one large β sheet composed of three antiparallel β strands. The Mg²⁺ binding site is located on the β sheet and consists of three amino acids, Asp-10, Glu-48, and Gly-11 (Katayanagi et al., Proteins: Struct., Funct., Genet., 1993, 17, 337-346). This structural motif of the Mg²⁺ binding site surrounded by β strands is similar to that in DNase I (Suck, D., and Oefner, C., Nature, 1986, 321, 620-625). The minor domain is believed to constitute the predominant binding region of the enzyme and is composed of an α helix terminating with a loop. The loop region is composed of a cluster of positively charged amino acids that are believed to bind electrostatistically to the minor groove of the DNA/RNA heteroduplex substrate. Although the conformation of the RNA/DNA substrate can vary from A-form to B-form depending on the sequence composition, in general RNA/DNA heteroduplexes adopt an A-like geometry (Pardi et al., Biochemistry, 1981, 20, 3986-3996; Hall, K. B., and Mclaughlin, L. W., Biochemistry, 1991, 30, 10606-10613; Lane et al., Eur. J. Biochem., 1993, 215, 297-306). The entire binding interaction appears to comprise a single helical turn of the substrate duplex. More recently, the binding characteristics, substrate requirements, cleavage products and effects of various chemical modifications of the substrates on the kinetic characteristics of E. coli RNase HI have been studied in more detail (Crooke, S. T. et al., Biochem. J., 1995, 312, 599-608; Lima, W. F. and Crooke, S. T., Biochemistry, 1997, 36, 390-398; Lima, W. F. et al., J. Biol. Chem., 1997, 272, 18191-18199; Tidd, D. M. and Worenius, H. M., Br. J. Cancer, 1989, 60, 343; Tidd, D. M. et al., Anti-Cancer Drug Des., 1988, 3, 117.

In addition to RNase HI, a second E. coli RNase H, RNase HII, has been cloned and characterized (Itaya, M., Proc. Natl. Acad. Sci. USA, 1990, 87, 8587-8591). It is comprised of 213 amino acids while RNase HI is 155 amino acids long. RNase HII displays only 17% homology with E. coli RNase HI. An RNase H cloned from S. typhimurium differed from E. coli RNase HI in only 11 positions and was 155 amino acids in length (Itaya, M. and Kondo K., Nucleic Acids Res., 1991, 19, 4443-4449; Itaya et al., Mol. Gen. Genet., 1991, 227, 438-445). An enzyme cloned from S. cerevisae was 30% homologous to E. coli RNase HI (Itaya, M. and Kondo K., Nucleic Acids Res., 1991, 19, 4443-4449; Itaya et al., Mol. Gen. Genet., 1991, 227, 438-445).

Proteins that display RNase H activity have also been cloned and purified from a number of viruses, other bacteria and yeast (Wintersberger, U. Pharmac. Ther., 1990, 48, 259-280). In many cases, proteins with RNase H activity appear to be fusion proteins in which RNase H is fused to the amino or carboxy end of another enzyme, often a DNA or RNA polymerase. The RNase H domain has been consistently found to be highly homologous to E. coli RNase HI, but because the other domains vary substantially, the molecular weights and other characteristics of the fusion proteins vary widely.

In higher eukaryotes two classes of RNase H have been defined based on differences in molecular weight, effects of divalent cations, sensitivity to sulfhydryl agents and immunological cross-reactivity (Busen et al., Eur. J. Biochem., 1977, 74, 203-208). RNase HII enzymes (also called RNases H2; formerly called Type 1 RNase H) are reported to have molecular weights in the 68-90 kDa range, be activated by either Mn²⁺ or Mg²⁺ and be insensitive to sulfhydryl agents. In contrast, RNase HI enzymes (also called RNases H1, formerly called Type 2 RNases H) have been reported to have molecular weights ranging from 31-45 kDa, to require Mg²⁺ to be highly sensitive to sulfhydryl agents and to be inhibited by Mn²⁺ (Busen, W., and Hausen, P., Eur. J. Biochem., 1975, 52, 179-190; Kane, C. M., Biochemistry, 1988, 27, 3187-3196; Busen, W., J. Biol. Chem., 1982, 257, 7106-7108.).

An enzyme with Type 2 RNase H characteristics has been purified to near homogeneity from human placenta (Frank et al., Nucleic Acids Res., 1994, 22, 5247-5254). This protein has a molecular weight of approximately 33 kDa and is active in a pH range of 6.5-10, with a pH optimum of 8.5-9. The enzyme requires Mg²⁺ and is inhibited by Mn²⁺ and n-ethyl maleimide. The products of cleavage reactions have 3′ hydroxyl and 5′ phosphate termini.

Multiple mammalian RNases H of each of at least two types have recently been cloned, expressed and sequenced. These include human RNase HI (also called Type 2 RNase H; Crooke et al., U.S. Pat. No. 6,001,653; Wu et al., Antisense Nucl. Acid Drug Des., 1998, 8, 53-61; Genbank Accession No. AF039652; Cerritelli and Crouch, 1998, genomics 53, 300-307; Frank et al., 1998, Biol. Chem. 379, 1407-1412, human RNase HII (also called Type 1 RNase H)(Frank et al., 1998, Proc. Natl. Acad. Sci. USA, 95, 12872-12877 and U.S. patent application Ser. No. 09/781, 712 filed Feb. 12, 2001, which claims, inter alia, a human RNase HII polypeptide prepared from a culture of ATCC Deposit No. PTA-2897, or mutant form or active fragment thereof) and other mammalian RNases H (Cerritelli and Crouch, ibid.,). The availability of purified RNase H has facilitated efforts to understand the structure of the enzyme, its distribution and the function(s) it may serve.

In addition to playing a natural role in DNA replication, RNase H has also been shown to be capable of cleaving the RNA component of certain oligonucleotide-RNA duplexes. While many mechanisms have been proposed for oligonucleotide mediated destabilization of target RNAs, the primary mechanism by which antisense oligonucleotides are believed to cause a reduction in target RNA levels is through this RNase H action. Monia et al., J. Biol. Chem., 1993, 266:13, 14514-14522. In vitro assays have demonstrated that oligonucleotides that are not substrates for RNase H can inhibit protein translation (Blake et al., Biochemistry, 1985, 24, 6139-4145) and that oligonucleotides inhibit protein translation in rabbit reticulocyte extracts that exhibit low RNase H activity. However, more efficient inhibition was found in systems that supported RNase H activity (Walder, R. Y. and Walder, J. A., Proc. Nat'l Acad. Sci. USA, 1988, 85, 5011-5015; Gagnor et al., Nucleic Acid Res., 1987, 15, 10419-10436; Cazenave et al., Nucleic Acid Res., 1989, 17, 4255-4273; and Dash et al., Proc. Nat'l Acad. Sci. USA, 1987, 84, 7896-7900.

DNA oligonucleotides having unmodified phosphodiester internucleoside linkages or modified phosphorothioate internucleoside linkages are substrates for cellular RNase H; i.e., they activate the cleavage of target RNA by the RNase H. (Dagle, J. M, Walder, J. A. and Weeks, D. L., Nucleic Acids Research 1990, 18, 4751; Dagle, J. M., Weeks, D. L. and Walder, J. A., Antisense Research And Development 1991, 1, 11; and Dagle, J. M., Andracki, M. E., DeVine, R. J. and Walder, J. A., Nucleic Acids Research 1991, 19, 1805). RNase H is an endonuclease that cleaves the RNA strand of RNA:DNA duplexes; activation of this enzyme therefore results in cleavage of the RNA target, and thus can greatly enhance the ability of antisense oligonucleotides to inhibit target RNA expression. Walder et al. note that in Xenopus embryos, both phosphodiester linkages and phosphorothioate linkages are also subject to exonuclease degradation. Such nuclease degradation is detrimental since it rapidly depletes the oligonucleotide available for RNase H activation. PCT Publication WO 89/05358, Walder et al., discloses DNA oligonucleotides modified at the 3′ terminal internucleoside linkage to make them resistant to nucleases while remaining substrates for RNase H.

Attempts to take advantage of the beneficial properties of oligonucleotide modifications while maintaining the substrate requirements for RNase H have led to the employment of chimeric oligonucleotides. Giles, R. V. et al., Anti-Cancer Drug Design 1992, 7, 37; Hayase, Y. et al., Biochemistry 1990, 29, 8793; Dagle, J. M. et al., Nucleic Acids Res. 1990, 18, 4751; Dagle, J. M. et al., Nucleic Acids Res., 1991, 19, 1805. Chimeric oligonucleotides contain two or more chemically distinct regions, each comprising at least one nucleotide. These oligonucleotides typically contain a region of modified nucleotides that confer one or more beneficial properties (such as, for example, increased nuclease resistance, increased uptake into cells, increased binding affinity for the RNA target) and an unmodified region that retains the ability to direct RNase H cleavage. This approach has been employed for a variety of backbone modifications, most commonly methylphosphonates, which alone are not substrates for RNase H. Methylphosphonate oligonucleotides containing RNase H-sensitive phosphodiester linkages were found to be able to direct target RNA cleavage by RNase H in vitro. Using E. coli RNase H, the minimum phosphodiester length required to direct efficient RNase H cleavage of target RNA strands has been reported to be either three or four linkages. Quartin, R. S. et al. Nucleic Acids Res. 1989, 17, 7253; Furdon, P. J. et al. Nucleic Acids Res. 1989, 17, 9193. Similar studies have been reported using in vitro mammalian RNase H cleavage assays. Agrawal, S. et al., Proc. Natl. Acad. Sci. USA 1990, 87, 1401. In this case, a series of backbone modifications, including methylphosphonates, containing different phosphodiester lengths were examined for cleavage efficiency. The minimum phosphodiester length required for efficient RNase H cleavage directed by oligonucleotides of this nature is five linkages. Subsequently, it has been shown that methylphosphonate/phosphodiester chimeras display increased specificity and efficiency for target RNA cleavage using E. coli RNase H in vitro. Giles, R. V. et al., Anti-Cancer Drug Design 1992, 7, 37. These compounds have also been reported to be effective antisense inhibitors in Xenopus oocytes and in cultured mammalian cells. Dagle, J. M. et al., Nucleic Acids Res. 1990, 18, 4751; Potts, J. D., et al., Proc. Natl. Acad. Sci. USA 1991, 88, 1516.

PCT Publication WO 90/15065, Froehler et al., discloses chimeric oligonucleotides “capped” at the 3′ and/or the 5′ end by phosphoramidite, phosphorothioate or phosphorodithioate linkages in order to provide stability against exonucleases while permitting RNase H activation. PCT Publication WO 91/12323, Pederson et al., discloses chimeric oligonucleotides in which two regions with modified backbones (methyl phosphonates, phosphoromorpholidates, phosphoropiperazidates or phosphoramidates) which do not activate RNase H flank a central deoxynucleotide region which does activate RNase H cleavage. 2′-deoxy oligonucleotides have been stabilized against nuclease degradation while still providing for RNase H activation by positioning a short section of phosphodiester linked nucleotides between sections of backbone-modified oligonucleotides having phosphoramidate, alkylphosphonate or phosphotriester linkages. Dagle, J. M, Walder, J. A. and Weeks, D. L., Nucleic Acids Research 1990, 18, 4751; Dagle, J. M., Weeks, D. L. and Walder, J. A., Antisense Research And Development 1991, 1, 11; and Dagle, J. M., Andracki, M. E., DeVine, R. J. and Walder, J. A., Nucleic Acids Research 1991, 19, 1805. While the phosphoramidate containing oligonucleotides were stabilized against exonucleases, each phosphoramidate linkage resulted in a loss of 1.6° C. in the measured Tₘ value of the phosphoramidate containing oligonucleotides. Dagle, J. M., Andracki, M. E., DeVine, R. J. and Walder, J. A., Nucleic Acids Research 1991, 19, 1805. Such loss of the Tₘ value is indicative of a decrease in the hybridization between the oligonucleotide and its target strand.

Saison-Behmoaras, T., Tocque, B. Rey, I., Chassignol, M., Thuong, N. T. and Helene, C., EMBO Journal 1991, 10, 1111, observed that even though an oligonucleotide was a substrate for RNase H, cleavage efficiency by RNase H was low because of weak hybridization to the mRNA.

Chimeric oligonucleotides are not limited to backbone modifications, although in the early 1990's, chimeric oligonucleotides containing 2′ ribose modifications mixed with RNase H-sensitive deoxy residues were not as well characterized as the backbone chimeras. EP Publication 260,032 (Inoue et al.) and Ohtsuka et al., FEBS Lett. 1987, 215, 327-330, employed 2′-O-methyl oligonucleotides (which alone would not be substrates for RNase H) containing unmodified deoxy gaps to direct cleavage in vitro by E. coli RNase H to specific sites within the complementary RNA strand. These compounds required a minimum deoxy gap of four bases for efficient target RNA cleavage. However, oligonucleotides of this nature were not examined for cleavage efficiency using mammalian RNase H nor tested for antisense activity in cells. These oligonucleotides were not stabilized against nucleases.

Studies on the ability to direct RNase H cleavage and antisense activity of 2′ ribose modifications other than O-methyl were, as of the early 1990's, extremely limited. Schmidt, S. et al., Biochim. Biophys. Acta 1992, 1130, 41.

While it has been recognized that cleavage of a target RNA strand using an antisense oligonucleotide and RNase H would be useful, nuclease resistance of the oligonucleotide and fidelity of the hybridization are also of great importance. There has been a long-felt need for methods or materials that could both activate RNase H while concurrently maintaining or improving hybridization properties and providing nuclease resistance. There remains a long-felt need for such methods and materials for enhancing antisense activity.

In the present invention, methods of using mammalian RNase H for reducing selected target RNA levels via an antisense mechanism are provided.

### SUMMARY OF THE INVENTION

The present invention provides methods of promoting antisense inhibition of expression of a target protein via use of mammalian RNase H, preferably RNase HI and/or RNase HII. The antisense oligonucleotide is a chimeric antisense oligonucleotide having a modification at the 2′ position of at least one sugar moiety. Preferably, the mammalian RNase H is a human RNase H.

Also provided are methods of eliciting cleavage of a selected cellular RNA target by contacting the RNA target with a chimeric antisense oligonucleotide having a modification at the 2′ position of at least one sugar moiety, allowing an oligonucleotide-RNA duplex to form, and contacting the duplex with mammalian RNase H under conditions in which cleavage of the RNA strand of the duplex occurs. Preferably the mammalian RNase H is an RNase HI or RNase HII. Also preferably, the mammalian RNase H is a human RNase H.

Further provided are methods for making an antisense oligonucleotide which elicits cleavage of its complementary target RNA by mammalian RNase H, wherein the oligonucleotide is a chimeric oligonucleotide which has a modification at the 2′ position of at least one sugar moiety. Preferably the mammalian RNase H is an RNase HI or RNase HII. Also preferably, the mammalian RNase H is a human RNase H.

### BRIEF DESCRIPTION OF THE DRAWINGS

FIG. 1 provides a novel human RNase HII primary sequence (299 amino acids; SEQ ID NO: 1) and sequence comparisons with mouse (SEQ ID NO: 2), C. elegans (SEQ ID NO: 3), yeast (300 amino acids; SEQ ID NO: 4) and E. coli RNase HII (298 amino acids; SEQ ID NO: 5). Boldface type indicates amino acid residues identical to human. Uppercase letters above alignment indicate amino acid residues identically conserved among species; lower case letters above alignment indicate residues similarly conserved.

FIG. 2 is a gel showing RNAse H dependent cleavage of complementary H-ras RNA by 2′-O-methyl chimeric phosphorothioate oligonucleotides. Lane designations refer to the length of the centered deoxy gap.

FIG. 3 is a two-part figure showing antisense activity of phosphorothioate 2′-O-methyl chimeric oligonucleotides targeted to ras codon-12 RNA sequences. FIG. 3A is a bar graph showing single-dose activity (100 nM) of uniform 2′-O-methyl oligonucleotides, uniform deoxy oligonucleotides and chimeric 2′-O-methyl oligonucleotides containing centered 1-, 3-, 5-, 7- or 9-base deoxy gaps. FIG. 3B is a line graph showing dose-response activity of uniform deoxy (▾) or 2′-O-methyl oligonucleotides containing centered 4-(▪,♦), 5-(●), 7-(+) or 9-base (▴) deoxy gaps.

FIG. 4 is a line graph showing correlation between antisense activity and ability to activate RNAse H as a function of deoxy gap length using phosphorothioate 2′-O-methyl oligonucleotides targeted against ras.

### DETAILED DESCRIPTION OF THE INVENTION

The present invention relates to methods for promoting antisense inhibition of a selected RNA target using mammalian RNase H, or for eliciting cleavage of a selected RNA target via antisense. In the context of this invention, “promoting antisense inhibition” or “promoting inhibition of expression” of a selected RNA target, or of its protein product, means inhibiting expression of the target or enhancing the inhibition of expression of the target. In one preferred embodiment, the mammalian RNase H is a human RNase H. The RNase H may be an RNase HI or an RNase HII. In one embodiment of these methods, the mammalian RNase H is present in an enriched amount. In the context of this invention, “enriched” means an amount greater than would naturally be found. RNase H may be present in an enriched amount through, for example, addition of exogenous RNase H, through selection of cells which overexpress RNase H or through manipulation of cells to cause overexpression of RNase H. The exogenously added RNase H may be added in the form of, for example, a cellular or tissue extract (such as HeLa cell extract), a biochemically purified or partially purified preparation of RNase H, or a cloned and expressed RNase H polypeptide.

The modulation of function of a target nucleic acid by compounds which specifically hybridize to it is generally referred to as “antisense”. The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is modulation of the expression of the target. In the context of the present invention, “modulation” means either an increase (stimulation) or a decrease (inhibition) in the expression of a gene. In the context of the present invention, inhibition is the preferred form of modulation of gene expression and mRNA is a preferred target.

It is preferred to target specific nucleic acids for antisense. “Targeting” an antisense compound to a particular nucleic acid, in the context of this invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. The targeting process also includes determination of a site or sites within this gene for the antisense interaction to occur such that the desired effect, e.g., detection or modulation of expression of the protein, will result. Within the context of the present invention, a preferred intragenic site is the region encompassing the translation initiation or termination codon of the open reading frame (ORF) of the gene. Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of the target, regardless of the sequence(s) of such codons.

It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which is known in the art to refer to the region between the translation initiation codon and the translation termination codon, is also a region which may be targeted effectively. Other target regions include the 5′ untranslated region (5′ UTR), known in the art to refer to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′ UTR), known in the art to refer to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The 5′ cap region may also be a preferred target region.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence. mRNA splice sites, i.e., intron-exon junctions, may also be preferred target regions, and are particularly useful in situations where aberrant splicing is implicated in disease, or where an overproduction of a particular mRNA splice product is implicated in disease. Aberrant fusion junctions due to rearrangements or deletions are also preferred targets. It has also been found that introns can also be effective, and therefore preferred, target regions for antisense compounds targeted, for example, to DNA or pre-mRNA.

Once one or more target sites have been identified, oligonucleotides are chosen which are sufficiently complementary to the target, i.e., hybridize sufficiently well and with sufficient specificity, to give the desired effect.

In the context of this invention, “hybridization” means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. “Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target. It is understood in the art that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired, i.e., under physiological conditions in the case of in vivo assays or therapeutic treatment, and in the case of in vitro assays, under conditions in which the assays are performed.

Antisense and other compounds of the invention which hybridize to the target and inhibit expression of the target are identified through experimentation, and the sequences of these compounds are hereinbelow identified as preferred embodiments of the invention. The target sites to which these preferred sequences are complementary are hereinbelow referred to as “active sites” and are therefore preferred sites for targeting. Therefore another embodiment of the invention encompasses compounds which hybridize to these active sites.

Antisense compounds are commonly used as research reagents and diagnostics. For example, antisense oligonucleotides, which are able to inhibit gene expression with exquisite specificity, are often used by those of ordinary skill to elucidate the function of particular genes. Antisense compounds are also used, for example, to distinguish between functions of various members of a biological pathway. Antisense modulation has, therefore, been harnessed for research use.

The specificity and sensitivity of antisense is also harnessed by those of skill in the art for therapeutic uses. Antisense oligonucleotides have been employed as therapeutic moieties in the treatment of disease states in animals and man. Antisense oligonucleotide drugs, including ribozymes, have been safely and effectively administered to humans and numerous clinical trials are presently underway. It is thus established that oligonucleotides can be useful therapeutic modalities that can be configured to be useful in treatment regimes for treatment of cells, tissues and animals, especially humans.

In the context of this invention, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

While antisense oligonucleotides are a preferred form of antisense compound, the present invention comprehends other oligomeric antisense compounds, including but not limited to oligonucleotide mimetics such as are described below. The antisense compounds in accordance with this invention preferably comprise from about 8 to about 50 nucleobases (i.e. from about 8 to about 50 linked nucleosides). Particularly preferred antisense compounds are antisense oligonucleotides, even more preferably those comprising from about 12 to about 30 nucleobases. Antisense compounds include ribozymes, external guide sequence (EGS) oligonucleotides (oligozymes), and other short catalytic RNAs or catalytic oligonucleotides which hybridize to the target nucleic acid and modulate its expression.

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

Specific examples of preferred antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e. a single inverted nucleoside residue which may be a basic (the nucleobase is missing or has a hydroxyl group in place thereof). Various salts, mixed salts and free acid forms are also included.

Representative United States patents that teach the preparation of the above phosphorus-containing linkages include, but are not limited to, U.S. Pat. Nos.: 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, certain of which are commonly owned with this application, and each of which is herein incorporated by reference.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos.: 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, certain of which are commonly owned with this application, and each of which is herein incorporated by reference.

In other preferred oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos.: 5,539,082; 5,714,331; and 5,719,262, each of which is herein incorporated by reference. Further teaching of PNA compounds can be found in Nielsen et al., Science, 1991, 254, 1497-1500.

Most preferred embodiments of the invention are oligonucleotides with phosphorothioate backbones and oligonucleosides with heteroatom backbones, and in particular —CH₂—NH—O—CH₂—, —CH₂—N(CH₃)—O—CH₂— [known as a methylene (methylimino) or MMI backbone], —CH₂—O—N(CH₃)—CH₂—, —CH₂—N(CH₃) —N(CH₃)—CH₂— and —O—N(CH₃)—CH₂—CH₂— [wherein the native phosphodiester backbone is represented as —O—P—O—CH₂— ] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further preferred modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂, also described in examples hereinbelow.

A further preferred modification includes Locked Nucleic Acids (LNAs) in which the 2′-hydroxyl group is linked to the 3′ or 4′ carbon atom of the sugar ring thereby forming a bicyclic sugar moiety. The linkage is preferably a methelyne (—CH₂—)ₙ group bridging the 2′ oxygen atom and the 3′ or 4′ carbon atom wherein n is 1 or 2. LNAs and preparation thereof are described in WO 98/39352 and WO 99/14226.

Other preferred modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. A preferred 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents that teach the preparation of such modified sugar structures include, but are not limited to, U.S. Pat. Nos.: 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, certain of which are commonly owned with the instant application, and each of which is herein incorporated by reference in its entirety.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′-O-methoxyethyl sugar modifications.

Representative United States patents that teach the preparation of certain of the above noted modified nucleobases as well as other modified nucleobases include, but are not limited to, the above noted U.S. Pat. No. 3,687,808, as well as U.S. Pat. Nos.: 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; and 5,681,941, certain of which are commonly owned with the instant application, and each of which is herein incorporated by reference, and U.S. Pat. No. 5,750,692, which is commonly owned with the instant application and also herein incorporated by reference.

Another modification of the oligonucleotides of the invention involves chemically linking to the oligonucleotide one or more moieties or conjugates which enhance the activity, cellular distribution or cellular uptake of the oligonucleotide. The compounds of the invention can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugates groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve oligomer uptake, enhance oligomer resistance to degradation, and/or strengthen sequence-specific hybridization with RNA. Groups that enhance the pharmacokinetic properties, in the context of this invention, include groups that improve oligomer uptake, distribution, metabolism or excretion. Representative conjugate groups are disclosed in International Patent Application PCT/US92/09196, filed Oct. 23, 1992 the entire disclosure of which is incorporated herein by reference. Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937. Oligonucleotides of the invention may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic. Oligonucleotide-drug conjugates and their preparation are described in U.S. patent application Ser. No. 09/334,130 (filed Jun. 15, 1999) which is incorporated herein by reference in its entirety.

Representative United States patents that teach the preparation of such oligonucleotide conjugates include, but are not limited to, U.S. Pat. Nos.: 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, certain of which are commonly owned with the instant application, and each of which is herein incorporated by reference.

It is not necessary for all positions in a given compound to be uniformly modified, and in fact more than one of the aforementioned modifications may be incorporated in a single compound or even at a single nucleoside within an oligonucleotide. The present invention preferably includes antisense compounds which are chimeric compounds. “Chimeric” antisense compounds or “chimeras,” in the context of this invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids.

By way of example, RNase H cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Oligonucleotides, particularly chimeric oligonucleotides, designed to elicit target cleavage by RNase H, thus are generally more potent than oligonucleotides of the same base sequence which are not so optimized. Cleavage of the RNA target can be routinely detected by, for example, gel electrophoresis and, if necessary, associated nucleic acid hybridization techniques known in the art.

Chimeric oligonucleotides may have one or more modifications of the internucleoside (backbone) linkage, the sugar or the base. In a preferred embodiment, the oligonucleotide is a chimeric oligonucleotide having a modification at the 2′ position of at least one sugar moiety. Presently believed to be particularly preferred are chimeric oligonucleotides which have approximately four or more deoxynucleotides in a row, which provide an RNase H cleavage site, flanked on one or both sides by a region of 2′-modified oligonucleotides.

Chimeric antisense compounds of the invention may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures include, but are not limited to, U.S. Pat. Nos.: 5,013,830; 5,149,797; 5,220,007; 5,256,775; 5,366,878; 5,403,711; 5,491,133; 5,565,350; 5,623,065; 5,652,355; 5,652,356; and 5,700,922, certain of which are commonly owned with the instant application, and each of which is herein incorporated by reference in its entirety.

RNase H, by definition, cleaves the RNA strand of an RNA-DNA duplex. In exploiting RNase H for antisense technology, the DNA portion of the duplex is generally an antisense oligonucleotide. Because native DNA oligonucleotides (2′ deoxy oligonucleotides with phosphodiester linkages) are relatively unstable in cells due to poor nuclease resistance, modified oligonucleotides are preferred for antisense. For example, oligodeoxynucleotides with phosphorothioate backbone linkages are often used. This is an example of a DNA-like oligonucleotide which is able to elicit RNase H cleavage of its complementary target RNA. Nucleic acid helices can adopt more than one type of structure, most commonly the A- and B-forms. It is believed that, in general, oligonucleotides which have B-form-like conformational geometry are “DNA-like” and will be able to elicit RNase H upon duplexation with an RNA target. Furthermore, oligonucleotides which contain a “DNA-like” region of B-form-like conformational geometry are also believed to be able to elicit RNase H upon duplexation with an RNA target. Thus in a gapped chimeric oligonucleotide or “gapmer,” it is preferred that the B-form portion be in the gap region (the region for eliciting RNase H cleavage).

The nucleotides for this B-form portion are selected to specifically include ribo-pentofuranosyl and arabinopentofuranosyl nucleotides. 2′-Deoxy-erythro-pentofuranosyl nucleotides also have B-form geometry and elicit RNase H activity. While not specifically excluded, if 2′-deoxy-erythro-pentofuranosyl nucleotides are included in the B-form portion of an oligonucleotide of the invention, such 2′-deoxy-erythro-pentofuranosyl nucleotides preferably do not constitute the totality of the nucleotides of that B-form portion of the oligonucleotide, but should be used in conjunction with ribonucleotides or an arabino nucleotides. As used herein, B-form geometry is inclusive of both C2′-endo and O4′-endo pucker, and the ribo and arabino nucleotides selected for inclusion in the oligonucleotide B-form portion are selected to be those nucleotides having C2′-endo conformation or those nucleotides having O4′-endo conformation. This is consistent with Berger, et. al., Nucleic Acids Research, 1998, 26, 2473-2480, who pointed out that in considering the furanose conformations in which nucleosides and nucleotides reside, B-form consideration should also be given to a O4′-endo pucker contribution.

Preferred for use as the B-form nucleotides for eliciting RNase H are ribonucleotides having 2′-deoxy-2′-S-methyl, 2′-deoxy-2′-methyl, 2′-deoxy-2′-amino, 2′-deoxy-2′-mono or dialkyl substituted amino, 2′-deoxy-2′-fluoromethyl, 2′-deoxy-2′-difluoromethyl, 2′-deoxy-2′-trifluoromethyl, 2′-deoxy-2′-methylene, 2′-deoxy-2′-fluoromethylene, 2′-deoxy-2′-difluoromethylene, 2′-deoxy-2′-ethyl, 2′-deoxy-2′-ethylene and 2′-deoxy-2′-acetylene. These nucleotides can alternately be described as 2′-SCH₃ ribonucleotide, 2′-CH₃ ribonucleotide, 2′-NH₂ ribonucleotide 2′-NH(C₁-C₂ alkyl) ribonucleotide, 2′-N(C₁-C₂ alkyl)₂ ribonucleotide, 2′-CH₂F ribonucleotide, 2′-CHF₂ ribonucleotide, 2′-CF₃ ribonucleotide, 2′═CH₂ ribonucleotide, 2′═CHF ribonucleotide, 2′═CF₂ ribonucleotide, 2′-C₂H₅ ribonucleotide, 2′-CH═CH₂ ribonucleotide, 2′-C≡CH ribonucleotide. A further useful ribonucleotide is one having a ring located on the ribose ring in a cage-like structure including 3′,O,4′-C-methyleneribonucleotides. Such cage-like structures will physically fix the ribose ring in the desired conformation.

Additionally, preferred for use as the B-form nucleotides for eliciting RNase H are arabino nucleotides having 2′-deoxy-2′-cyano, 2′-deoxy-2′-fluoro, 2′-deoxy-2′-chloro, 2′-deoxy-2′-bromo, 2′-deoxy-2′-azido, 2′-methoxy and the unmodified arabino nucleotide (that includes a 2′-OH projecting upwards towards the base of the nucleotide). These arabino nucleotides can alternately be described as 2′-CN arabino nucleotide, 2′-F arabino nucleotide, 2′-Cl arabino nucleotide, 2′-Br arabino nucleotide, 2′-N₃ arabino nucleotide, 2′-O—CH₃ arabino nucleotide and arabino nucleotide.

Such nucleotides are linked together via phosphorothioate, phosphorodithioate, boranophosphate or phosphodiester linkages. particularly preferred is the phosphorothioate linkage.

Illustrative of the B-form nucleotides for use in the invention is a 2′-S-methyl (2′-SMe) nucleotide that resides in C2′ endo conformation. It has been compared by molecular modeling to a 2′-O-methyl (2′-OMe)nucleotide that resides in a C3′ endo conformation.

The antisense compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

The following nonlimiting examples are provided to further illustrate the present invention.

### EXAMPLES

### Example 1

### Chimeric 2′-O-methyl Antisense Oligonucleotides with Deoxy Gaps, Targeted to Activated H-ras

Mutation of the ras gene, causing an amino acid alteration at one of three critical positions (one of which is codon 12) in the protein product, results in conversion to a form which is implicated in tumor formation. A gene having such a mutation is said to be “activated.” It is thought that such a point mutation leading to ras activation can be induced by carcinogens or other environmental factors. Overall, some 10 to 20% of human tumors have a mutation in one of the three ras genes (H-ras, K-ras, or N-ras). Oligonucleotides targeted to the H-ras codon-12 point mutation were effective in inhibiting expression of a ras-luciferase reporter gene system. A series of eleven phosphorothioate oligonucleotides, ranging in length between 5 and 25 bases, were made and tested for ability to inhibit mutant and wild type ras-luciferase in transient transfection assays. Based on the sequence of a mutant-selective 17-mer antisense oligonucleotide targeted to codon 12 of H-ras (CCACACCGACGGCGCCC; ISIS 2570; SEQ ID NO: 6), a series of chimeric phosphorothioate 2′-O-methyl oligonucleotides were synthesized in which the end regions consisted of 2′-O-methyl nucleosides and the central residues formed a “deoxy gap”. The number of deoxy residues ranged from zero (full 2′-O-methyl) to 17 (full deoxy). These oligonucleotides are shown in Table 1.

TABLE 1
Chimeric phosphorothioate oligonucleotides
having 2′-O-methyl ends (bold) and central deoxy gap
(Mutant codon-12 target)

| | | | SEQ |
|---|---|---|---|
| OLIGO | # | | ID |
| NO. | DEOXY | SEQUENCE | NO. |
| 4122 | 0 | CCACACCGACGGCGCCC | 6 |
| 3975 | 1 | CCACACCGACGGCGCCC | 6 |
| 3979 | 3 | CCACACCGACGGCGCCC | 6 |
| 4236 | 4 | CCACACCGACGGCGCCC | 6 |
| 4242 | 4 | CCACACCGACGGCGCCC | 6 |
| 3980 | 5 | CCACACCGACGGCGCCC | 6 |
| 3985 | 7 | CCACACCGACGGCGCCC | 6 |
| 3984 | 9 | CCACACCGACGGCGCCC | 6 |
| 2570 | 17 | CCACACCGACGGCGCCC | 6 |

### Example 2

### RNase H Analysis using E. coli Extract

The oligonucleotides shown in Table 1 were characterized for their ability to direct RNase H cleavage in vitro using E. coli extract as a source for mammalian RNase H, and for antisense activity. RNase H assays were performed using a chemically synthesized 25-base oligoribonucleotide corresponding to bases +23 to +47 of activated (codon 12, G→U) H-ras mRNA. The 5′ end-labeled RNA was used at a concentration of 20 nM and incubated with a 10-fold molar excess of antisense oligonucleotide in a reaction containing 20 mM tris-Cl, pH 7.5, 100 mM KCl, 10 mM MgCl₂, 1 mM dithiothreitol, 10 μg tRNA and 4 U RNasin in a final volume of 10 μl. The reaction components were preannealed at 37° C. for 15 minutes then allowed to cool slowly to room temperature. HeLa cell nuclear extracts were used as a source of mammalian RNase H. Reactions were initiated by addition of 2 μg of nuclear extract (5 μl) and reactions were allowed to proceed for 10 minutes at 37° C. Reactions were stopped by phenol/chloroform extraction and RNA components were precipitated with ethanol. Equal CPMs were loaded on a 20% polyacrylamide gel containing 7M urea and RNA cleavage products were resolved and visualized by electrophoresis followed by autoradiography. Quantitation of cleavage products was performed using a Molecular Dynamics Densitometer.

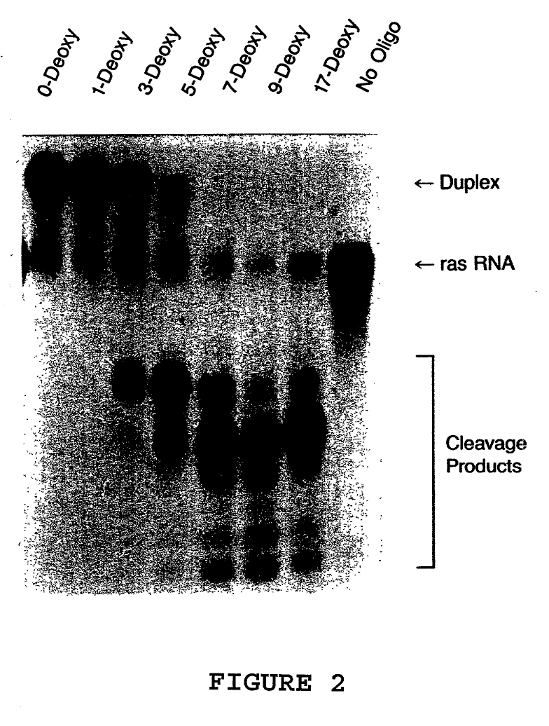

As shown in FIG. 2, no cleavage was observed with the fully modified 2′-O-methyl oligonucleotide or one containing a single deoxy residue. Oligonucleotides with a deoxy length of three, four, five, seven or nine were able to direct RNase H cleavage. Deoxy gaps of five, seven or nine are preferred and gaps of seven or nine are most preferred.

### Example 3

### Determination of Antisense Activity of Chimeric Oligonucleotides using a ras Transactivation Reporter Gene System

The oligonucleotides in Table 1 were tested for antisense activity against full length H-ras mRNA using a transient co-transfection reporter gene system in which H-ras gene expression was monitored using a ras-responsive enhancer element linked to the reporter gene luciferase. The expression plasmid pSV2-oli, containing an activated (codon 12, GGC→GTC) H-ras cDNA insert under control of the constitutive SV40 promoter, was a gift from Dr. Bruno Tocque (Rhone-Poulenc Sante, Vitry, France). This plasmid was used as a template to construct, by PCR, a H-ras expression plasmid under regulation of the steroid-inducible mouse mammary tumor virus (MMTV) promoter. To obtain H-ras coding sequences, the 570 bp coding region of the H-ras gene was amplified by PCR. The PCR primers were designed with unique restriction endonuclease sites in their 5′-regions to facilitate cloning. The PCR product containing the coding region of the H-ras codon 12 mutant oncogene was gel purified, digested, and gel purified once again prior to cloning. This construction was completed by cloning the insert into the expression plasmid pMAMneo (Clontech Laboratories, CA). The ras-responsive reporter gene pRDO53 was used to detect ras expression. Owen et al., Proc. Natl. Acad. Sci. U.S.A. 1990, 87, 3866-3870.

The ras-luciferase reporter genes described in this study were assembled using PCR technology. Oligonucleotide primers were synthesized for use as primers for PCR cloning of the 5′-regions of exon 1 of both the mutant (codon 12) and non-mutant (wild-type) human H-ras genes. The plasmids pT24-C3, containing the c-H-ras1 activated oncogene (codon 12, GGC→GTC), and pbc-N1, containing the c-H-ras protooncogene, were obtained from the American Type Culture Collection (Bethesda, Md.). The plasmid pT3/T7 luc, containing the 1.9 kb firefly luciferase gene, was obtained from Clontech Laboratories (Palo Alto, Calif.). The oligonucleotide PCR primers were used in standard PCR reactions using mutant and non-mutant H-ras genes as templates. These primers produce a DNA product of 145 base pairs corresponding to sequences −53 to +65 (relative to the translational initiation site) of normal and mutant H-ras, flanked by NheI and HindIII restriction endonuclease sites. The PCR product was gel purified, precipitated, washed and resuspended in water using standard procedures.

PCR primers for the cloning of the P. pyralis (firefly) luciferase gene were designed such that the PCR product would code for the full-length luciferase protein with the exception of the amino-terminal methionine residue, which would be replaced with two amino acids, an amino-terminal lysine residue followed by a leucine residue. The oligonucleotide PCR primers used for the cloning of the luciferase gene were used in standard PCR reactions using a commercially available plasmid (pT3/T7-Luc) (Clontech), containing the luciferase reporter gene, as a template. These primers yield a product of approximately 1.9 kb corresponding to the luciferase gene, flanked by unique HindIII and BssHII restriction endonuclease sites. This fragment was gel purified, precipitated, washed and resuspended in water using standard procedures.

To complete the assembly of the ras-luciferase fusion reporter gene, the ras and luciferase PCR products were digested with the appropriate restriction endonucleases and cloned by three-part ligation into an expression vector containing the steroid-inducible mouse mammary tumor virus promotor MMTV using the restriction endonucleases NheI, HindIII and BssHII. The resulting clone results in the insertion of H-ras 5′ sequences (−53 to +65) fused in frame with the firefly luciferase gene. The resulting expression vector encodes a ras-luciferase fusion product which is expressed under control of the steroid-inducible MMTV promoter. These plasmid constructions contain sequences encoding amino acids 1-22 of activated (RA2) or normal (RA4) H-ras proteins fused in frame with sequences coding for firefly luciferase. Translation initiation of the ras-luciferase fusion mRNA is dependent upon the natural H-ras AUG codon. Both mutant and normal H-ras luciferase fusion constructions were confirmed by DNA sequence analysis using standard procedures.

Cells were transfected with plasmid DNA as described by Greenberg, M. E., in Current Protocols in Molecular Biology, (F. M. Ausubel, R. Brent, R. E. Kingston, D. D. Moore, J. A. Smith, J. G. Seidman and K. Strahl, eds.), John Wiley and Sons, NY, with the following modifications. HeLa cells were plated on 60 mm dishes at 5×10⁵ cells/dish. A total of 10 μg or 12 μg of DNA was added to each dish, of which 1 μg was a vector expressing the rat glucocorticoid receptor under control of the constitutive Rous sarcoma virus (RSV) promoter and the remainder was ras-luciferase reporter plasmid. Calcium phosphate-DNA coprecipitates were removed after 16-20 hours by washing with Tris-buffered saline [50 Mm Tris-Cl (pH 7.5), 150 mM NaCl] containing 3 mM EGTA. Fresh medium supplemented with 10% fetal bovine serum was then added to the cells. At this time, cells were pre-treated with antisense oligonucleotides prior to activation of reporter gene expression by dexamethasone.

Following plasmid transfection, cells were washed with phosphate buffered saline prewarmed to 37° C. and Opti-MEM containing 5 μg/mL N-[1-(2,3-dioleyloxy)propyl]-N,N,N,-trimethylammonium chloride (DOTMA) was added to each plate (1.0 ml per well). Oligonucleotides were added from 50 μM stocks to each plate and incubated for 4 hours at 37° C. Medium was removed and replaced with DMEM containing 10% fetal bovine serum and the appropriate oligonucleotide at the indicated concentrations and cells were incubated for an additional 2 hours at 37° C. before reporter gene expression was activated by treatment of cells with dexamethasone to a final concentration of 0.2 μM. Cells were harvested and assayed for luciferase activity fifteen hours following dexamethasone stimulation.

Luciferase was extracted from cells by lysis with the detergent Triton X-100 as described by Greenberg, M. E., in Current Protocols in Molecular Biology, (F. M. Ausubel, R. Brent, R .E. Kingston, D. D. Moore, J. A. Smith, J. G. Seidman and K. Strahl, eds.), John Wiley and Sons, NY. A Dynatech ML1000 luminometer was used to measure peak luminescence upon addition of luciferin (Sigma) to 625 μM. For each extract, luciferase assays were performed multiple times, using differing amounts of extract to ensure that the data were gathered in the linear range of the assay.

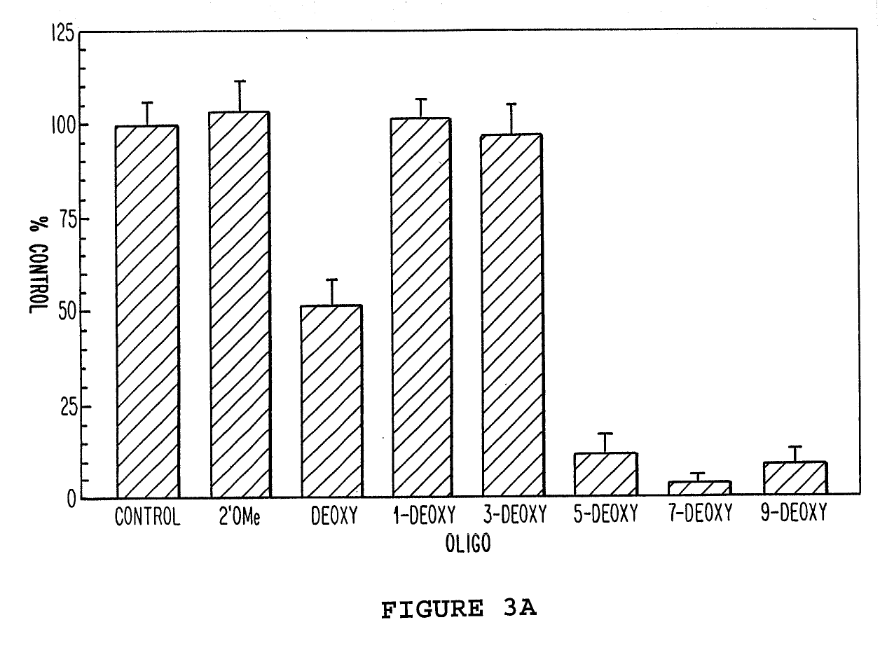

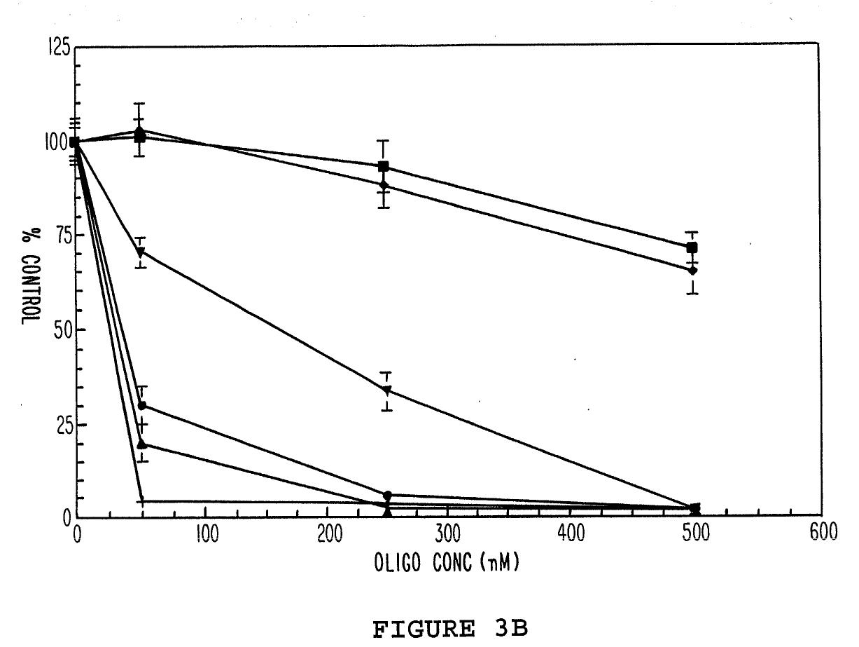

Antisense experiments were performed initially at a single oligonucleotide concentration (100 nM). As shown in FIG. 3A, chimeric 2′-O-methyl oligonucleotides containing deoxy gaps of five or more residues inhibited H-ras gene expression. These compounds displayed activities greater than that of the full deoxy parent compound. Thus the beneficial properties of enhanced target affinity conferred by 2′-O-methyl modifications can be exploited for antisense inhibition provided these compounds are equipped with RNase H-sensitive deoxy gaps of the appropriate length.

Dose response experiments were performed using these active compounds, along with the 2′-O-methyl chimeras containing four deoxy residues. As shown in FIG. 3B, oligonucleotide-mediated inhibition of full-length H-ras by these oligonucleotides was dose-dependent. The most active compound was the seven-residue deoxy chimera, which displayed an activity approximately five times greater than that of the full deoxy oligonucleotide.

The substrate requirements of RNase H can also be exploited to obtain selectivity for a target sequence compared to one with a mismatched base (for example, to distinguish a target bearing single base mutation such as that found at codon 12 of activated H-ras from the wild-type target sequence). If the enzyme is unable to bind or cleave a mismatch, additional selectivity will be obtained beyond that conferred by normal mismatch hybridization characteristics (ΔΔG°₃₇), by employing chimeric oligonucleotides that place the RNase H recognition site at the mismatch. This has been found to be the case; RNase H can indeed discriminate between a fully matched duplex and one containing a single mismatch.

### Example 4

### Shortened Chimeric Oligonucleotides

Enhanced target affinity conferred by the 2′-O-methyl modifications was found to confer activity on short chimeric oligonucleotides. A series of short (11, 13, 15 and 17-mer) 2′-O-methyl chimeric oligonucleotides (shown in Table 2) were tested for antisense activity vs. full length ras again using the luciferase reporter assay. In sharp contrast to the full deoxy 13-mer, both 2′-O-methylchimeric 13-mers inhibited ras expression, and one of the 11-mers was also active.

TABLE 2
Shortened chimeric oligonucleotides targeted to human ras

| LENGTH | SEQUENCE | SEQ ID NO: |
|---|---|---|
| 17 | CCACACCGACGGCGCCC | 6 |
| 15 | CACACCGACGGCGCC | 7 |
| 13 | ACACCGACGGCGC | 8 |
| 11 | CACCGACGGCG | 9 |
| 17 | CCACACCGACGGCGCCC | 6 |
| 15 | CACACCGACGGCGCC | 7 |
| 13 | ACACCGACGGCGC | 8 |
| 11 | CACCGACGGCG | 9 |

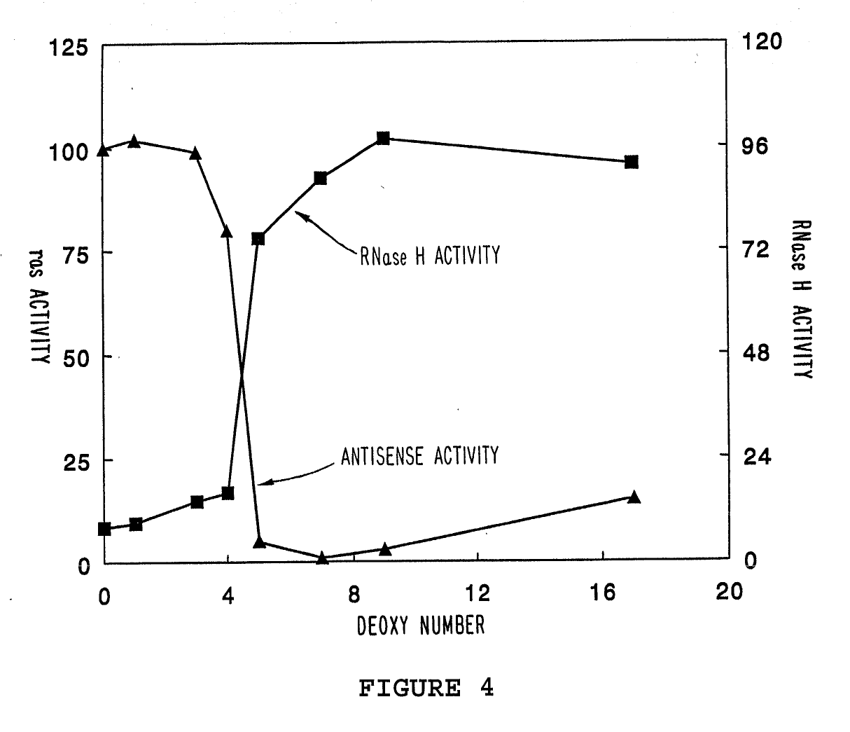

Relative antisense activity and ability to activate RNase H cleavage in vitro by chimeric 2′-O-methyl oligonucleotides is well correlated with deoxy length, as shown in FIG. 4).

### Example 5

### Asymmetrical Deoxy Gaps

It is not necessary that the deoxy gap be in the center of the chimeric molecule. It was found that chimeric molecules having the nucleotides of the region at one end modified at the 2′ position to enhance binding and the remainder of the molecule unmodified (2′ deoxy) can still inhibit ras expression. Oligonucleotides of SEQ ID NO: 6 (17-mer complementary to mutant codon 12) in which a 7-deoxy gap was located at either the 5′ or 3′ side of the 17-mer, or at different sites within the middle of the molecule, all demonstrated RNase H activation and antisense activity. However, a 5-base gap was found to be more sensitive to placement, as some gap positions rendered the duplex a poor activator of RNase H and a poor antisense inhibitor. Therefore, a 7-base deoxy gap is preferred.

### Example 6

### Other Sugar Modifications

The effects of other 2′ sugar modifications besides 2′-O-methyl on antisense activity in chimeric oligonucleotides have been examined. These modifications are listed in Table 3.

TABLE 3
2′-modified 17-mer with 7-deoxy gap
CCACACCGACGGCGCCC (SEQ ID NO: 6)

| 2′ MODIFICATION | IC50 (nM) |
|---|---|
| -Deoxy | 150 |
| —O-Pentyl | 150 |
| —O-Propyl | 70 |
| —O-Methyl | 20 |
| -Fluoro | 10 |

These 2′ modified oligonucleotides were tested for antisense activity against H-ras using the transactivation reporter gene assay. As shown in Table 3, all of these 2′ modified chimeric compounds inhibited ras expression, with the 2′-fluoro 7-deoxy-gap compound the most active. A 2′-fluoro chimeric oligonucleotide with a centered 5-deoxy gap was also active.

Chimeric phosphorothioate oligonucleotides having SEQ ID NO: 6 having 2′-O-propyl regions surrounding a 5-base or 7-base deoxy gap were compared to 2′-O-methyl chimeric oligonucleotides. ras expression in T24 cells was inhibited by both 2′-O-methyl and 2′-O-propyl chimeric oligonucleotides with a 7-deoxy gap and a uniform phosphorothioate backbone. When the deoxy gap was decreased to five nucleotides, only the 2′-O-methyl oligonucleotide inhibited ras expression.

### Example 7

### Antisense Oligonucleotide Inhibition of H-ras Gene Expression in Cancer Cells

Oligonucleotide 2570, a 17-mer phosphorothioate oligonucleotide complementary to the codon 12 region of activated H-ras, was tested for inhibition of ras expression in T24 cells along with chimeric phosphorothioate 2′-O-methyl oligonucleotides 3980, 3985 and 3984, which have the same sequence as 2570 and have deoxy gaps of 5, 7 and 9 bases, respectively (shown in Table 1).

The human urinary bladder cancer cell line T24 was obtained from the American Type Culture Collection (Rockville Md.). Cells were grown in McCoy's 5A medium with L-glutamine (Gibco BRL, Gaithersburg Md.), supplemented with 10% heat-inactivated fetal calf serum and 50 U/ml each of penicillin and streptomycin. Cells were seeded on 100 mm plates. When they reached 70% confluency, they were treated with oligonucleotide. Plates were washed with 10 ml prewarmed PBS and 5 ml of Opti-MEM reduced-serum medium containing 2.5 μl DOTMA. oligonucleotide was then added to the desired concentration. After 4 hours of treatment, the medium was replaced with McCoy's medium. Cells were harvested 48 hours after oligonucleotide treatment and RNA was isolated using a standard CsCl purification method. Kingston, R. E., in Current Protocols in Molecular Biology, (F. M. Ausubel, R. Brent, R. E. Kingston, D. D. Moore, J. A. Smith, J. G. Seidman and K. Strahl, eds.), John Wiley and Sons, NY.

The human epithelioid carcinoma cell line HeLa 229 was obtained from the American Type Culture Collection (Bethesda, Md.). HeLa cells were maintained as monolayers on 6-well plates in Dulbecco's Modified Eagle's medium (DMEM) supplemented with 10% fetal bovine serum and 100 U/ml penicillin. Treatment with oligonucleotide and isolation of RNA were essentially as described above for T24 cells.

Northern hybridization: 10 μg of each RNA was electrophoresed on a 1.2% agarose/formaldehyde gel and transferred overnight to GeneBind 45 nylon membrane (Pharmacia LKB, Piscataway, N.J.) using standard methods. Kingston, R. E., in Current Protocols in Molecular Biology, (F. M. Ausubel, R. Brent, R. E. Kingston, D. D. Moore, J. A. Smith, J. G. Seidman and K. Strahl, eds.), John Wiley and Sons, NY. RNA was UV-crosslinked to the membrane. Double-stranded ³²P-labeled probes were synthesized using the Prime a Gene labeling kit (Promega, Madison Wis.). The ras probe was a SalI-NheI fragment of a cDNA clone of the activated (mutant) H-ras mRNA having a GGC-to-GTC mutation at codon-12. The control probe was G3PDH. Blots were prehybridized for 15 minutes at 68° C. with the QuickHyb hybridization solution (Stratagene, La Jolla, Calif.). The heat-denatured radioactive probe (2.5×10⁶ counts/2 ml hybridization solution) mixed with 100 μl of 10 mg/ml salmon sperm DNA was added and the membrane was hybridized for 1 hour at 68° C. The blots were washed twice for 15 minutes at room temperature in 2×SSC/0.1% SDS and once for 30 minutes at 60° C. with 0.1×SSC/0.1% SDS. Blots were autoradiographed and the intensity of signal was quantitated using an ImageQuant PhosphorImager (Molecular Dynamics, Sunnyvale, Calif.). Northern blots were first hybridized with the ras probe, then stripped by boiling for 15 minutes in 0.1×SSC/0.1% SDS and rehybridized with the control G3PDH probe to check for correct sample loading. The fully 2′-deoxy oligonucleotide 2570 and the three chimeric oligonucleotides decreased ras mRNA levels in T24 cells. Compounds 3985 (7-deoxy gap) and 3984 (9-deoxy gap) decreased ras mRNA by 81%; compound 3980 (5-deoxy gap) decreased ras mRNA by 61%. Chimeric oligonucleotides having this sequence, but having 2′-fluoro-modified nucleotides flanking a 5-deoxy (4689) or 7-deoxy (4690) gap, inhibited ras mRNA expression in T24 cells, with the 7-deoxy gap being preferred (82% inhibition, vs 63% inhibition for the 2′-fluoro chimera with a 5-deoxy gap).

### Example 8

### Antisense Oligonucleotide Inhibition of Proliferation of Cancer Cells

Three 17-mer oligonucleotides having the same sequence (SEQ ID NO: 6), complementary to the codon 12 region of activated ras, were tested for effects on T24 cancer cell proliferation. 3985 has a 7-deoxy gap flanked by 2′-O-methyl nucleotides, and 4690 has a 7-deoxy gap flanked by 2′-F nucleotides (all are phosphorothioates). Cells were cultured and treated with oligonucleotide essentially as described in the previous example. Cells were seeded on 60 mm plates and were treated with oligonucleotide in the presence of DOTMA when they reached 70% confluency. Time course experiment: On day 1, cells were treated with a single dose of oligonucleotide at a final concentration of 100 nM. The growth medium was changed once on day 3 and cells were counted every day for 5 days, using a counting chamber. Dose-response experiment: Various concentrations of oligonucleotide (10, 25, 50, 100 or 250 nM) were added to the cells and cells were harvested and counted 3 days later. Oligonucleotides 2570, 3985 and 4690 were tested for effects on T24 cancer cell proliferation. Effects of these oligonucleotides on cancer cell proliferation correlated well with their effects on ras mRNA expression shown by Northern blot analysis: oligonucleotide 2570 inhibited cell proliferation by 61%, the 2′-O-methyl chimeric oligonucleotide 3985 inhibited cell proliferation by 82%, and the 2′-fluoro chimeric analog inhibited cell proliferation by 93%.

In dose-response studies of these oligonucleotides on cell proliferation, the inhibition was shown to be dose-dependent in the 25 nM-100 nM range. IC50 values of 44 nM, 61 nM and 98 nM could be assigned to oligonucleotides 4690, 3985 and 2570, respectively. The random oligonucleotide control had no effect at the doses tested.

The effect of ISIS 2570 on cell proliferation was cell type-specific. The inhibition of T24 cell proliferation by this oligonucleotide was four times as severe as the inhibition of HeLa cells by the same oligonucleotide (100 nM oligonucleotide concentration). ISIS 2570 is targeted to the activated (mutant) ras codon 12, which is present in T24 but lacking in HeLa cells, which have the wild-type codon 12.

### Cloning and Expression of Human RNase HI

### Example 9

### Rapid Amplification of 5′-cDNA end (5′ -RACE) and 3′-cDNA end (3′-RACE)

An Internet search of the XREF database in the National Center of Biotechnology Information (NCBI) yielded a 361 base pair (bp) human expressed sequenced tag (EST, GenBank accession #H28861), homologous to yeast RNase H (RNH1) protein sequenced tag (EST, GenBank accession #Q04740) and its chicken homologue (accession #D26340). Three sets of oligonucleotide primers encoding the human RNase H EST sequence were synthesized. The sense primers were ACGCTGGCCGGGAGTCGAAATGCTTC (H1: SEQ ID NO: 10), CTGTTCCTGGCCCACAGAGTCGCCTTGG (H3: SEQ ID NO: 11) and GGTCTTTCTGACCTGGAATGAGTGCAGAG (H5: SEQ ID NO: 12). The antisense primers were CTTGCCTGGTTTCGCCCTCCGATTCTTGT (H2: SEQ ID NO: 13), TTGATTTTCATGCCCTTCTGAAACTTCCG (H4; SEQ ID NO: 14) and CCTCATCCTCTATGGCAAACTTCTTAAATCTGGC (H6; SEQ ID NO: 15). The human RNase H 3′ and 5′ cDNAs derived from the EST sequence were amplified by polymerase chain reaction (PCR), using human liver or leukemia (lymphoblastic Molt-4) cell line Marathon ready cDNA as templates, H1 or H3/AP1 as well as H4 or H6/AP2 as primers (Clontech, Palo Alto, Calif.). The fragments were subjected to agarose gel electrophoresis and transferred to nitrocellulose membrane (Bio-Rad, Hercules Calif.) for confirmation by Southern blot, using ³²P-labeled H2 and H1 probes (for 3′ and 5′ RACE products, respectively, in accordance with procedures described by Ausubel et al., Current Protocols in Molecular Biology, Wiley and Sons, New York, N.Y., 1988. The confirmed fragments were excised from the agarose gel and purified by gel extraction (Qiagen, Germany), then subcloned into Zero-blunt vector (Invitrogen, Carlsbad, Calif.) and subjected to DNA sequencing.

### Example 10

### Screening of the cDNA Library, Human RNase HI DNA Sequencing and Sequence Analysis

A human liver cDNA lambda phage Uni-ZAP library (Stratagene, La Jolla, Calif.) was screened using the RACE products as specific probes. The positive cDNA clones were excised into the pBluescript phagemid (Stratagene, La Jolla Calif.) from lambda phage and subjected to DNA sequencing with an automatic DNA sequencer (Applied Biosystems, Foster City, Calif.) by Retrogen Inc. (San Diego, Calif.). The overlapping sequences were aligned and combined by the assembling programs of MacDNASIS v3.0 (Hitachi Software Engineering America, South San Francisco, Calif.). Protein structure and subsequence analysis were performed by the program of Macvector 6.0 (Oxford Molecular Group Inc., Campbell, Calif.).

### Example 11

### Northern Blot and Southern Blot Analysis

Total RNA from different human cell lines (ATCC, Rockville, Md.) was prepared and subjected to formaldehyde agarose gel electrophoresis in accordance with procedures described by Ausubel et al., Current Protocols in Molecular Biology, Wiley and Sons, New York, N.Y., 1988, and transferred to nitrocellulose membrane (Bio-Rad, Hercules Calif.). Northern blot hybridization was carried out in QuickHyb buffer (Stratagene, La Jolla, Calif.) with ³²P-labeled probe of full length RNase H cDNA clone or primer H1/H2 PCR-generated 322-base N-terminal RNase H cDNA fragment at 68° C. for 2 hours. The membranes were washed twice with 0.1% SSC/0.1% SDS for 30 minutes and subjected to auto-radiography. Southern blot analysis was carried out in 1×pre-hybridization/hybridization buffer (BRL, Gaithersburg, Md.) with a labeled 430 bp C-terminal restriction enzyme PstI/PvuII fragment or 1.7 kb full length cDNA probe at 60° C. for 18 hours. The membranes were washed twice with 0.1% SSC/0.1% SDS at 60° C. for 30 minutes, and subjected to autoradiography.

### Example 12

### Expression and Purification of the Cloned Human RNase HI Protein

The cDNA fragment coding the full RNase H protein sequence was amplified by PCR using 2 primers, one of which contains restriction enzyme NdeI site adapter and six histidine (his-tag) codons and 22 bp protein N terminal coding sequence. The fragment was cloned into expression vector pET17b (Novagen, Madison, Wis.) and confirmed by DNA sequencing. The plasmid was transfected into E. coli BL21 (DE3) (Novagen, Madison, Wis.). The bacteria were grown in M9ZB medium at 32° C. and harvested when the OD₆₀₀ of the culture reached 0.8, in accordance with procedures described by Ausubel et al., Current Protocols in Molecular Biology, Wiley and Sons, New York, N.Y., 1988. Cells were lysed in 8M urea solution and recombinant protein was partially purified with Ni-NTA agarose (Qiagen, Germany). Further purification was performed with C4 reverse phase chromatography (Beckman, System Gold, Fullerton, Calif.) with 0.1% TFA water and 0.1% TFA acetonitrile gradient of 0% to 80% in 40 minutes as described by Deutscher, M. P., Guide to Protein Purification, Methods in Enzymology 182, Academic Press, New York, N.Y., 1990. The recombinant proteins and control samples were collected, lyophilized and subjected to 12% SDS-PAGE as described by Ausubel et al. (1988) Current Protocols in Molecular Biology, Wiley and Sons, New York, N.Y. The purified protein and control samples were resuspended in 6 M urea solution containing 20 mM Tris HCl, pH 7.4, 400 mM NaCl, 20% glycerol, 0.2 mM PMSF, 5 mM DTT, 10 μg/ml aprotinin and leupeptin, and refolded by dialysis with decreasing urea concentration from 6 M to 0.5 M as well as DTT concentration from 5 mM to 0.5 mM as described by Deutscher, M. P., Guide to Protein Purification, Methods in Enzymology 182, Academic Press, New York, N.Y., 1990. The refolded proteins were concentrated (10 fold) by Centricon (Amicon, Danvers, Mass.) and subjected to RNase H activity assay.

### Cloning and Expression of Human RNase HII

### Example 13

### Cloning Human RNase HII by Rapid Amplification of 5′-cDNA end (5′-RACE) and 3′-cDNA end (3′-RACE) of Human RNase HII

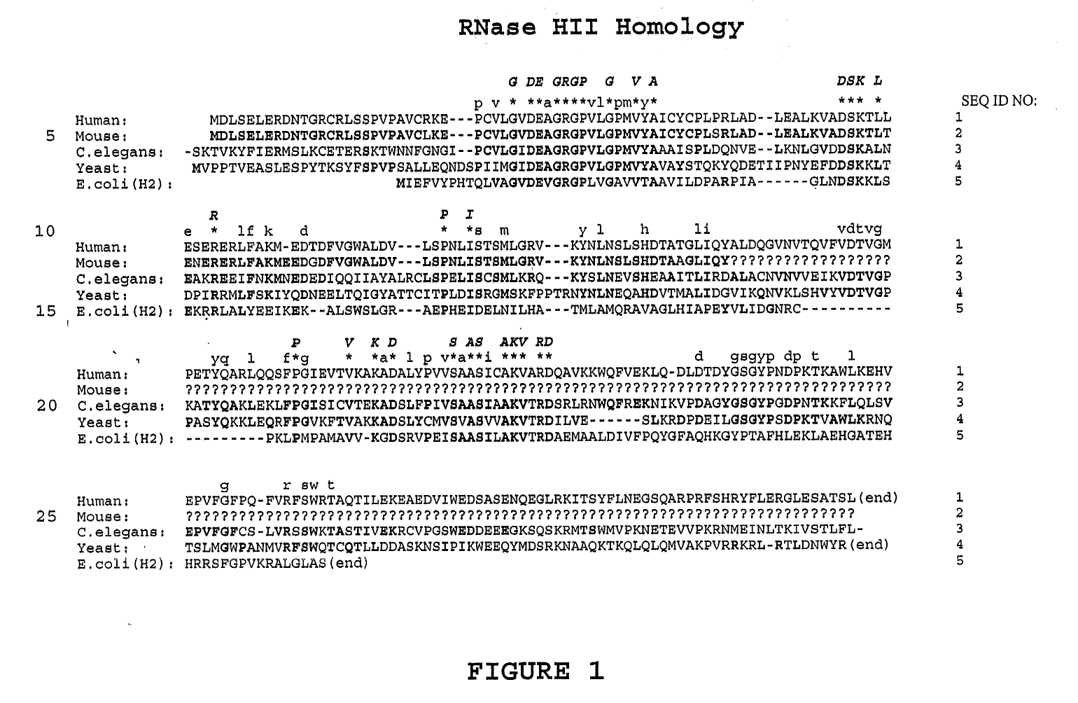

An internet search of the XREF database in the National Center of Biotechnology Information (NCBI) yielded 2 overlapping human expressed sequence tags (ESTs), GenBank accession numbers W05602 and H43540, homologous to yeast RNase HII (RNH2) protein sequence (GenBank accession number Z71348; SEQ ID NO: 4 shown in FIG. 1), and its C. elegans homologue (accession number Z66524, of which amino acids 396-702 of gene TI3H5.2 correspond to SEQ ID NO: 3 shown in FIG. 1). Three sets of oligonucleotide primers hybridizable to one or both of the human RNase HII EST sequences were synthesized. The sense primers were AGCAGGCGCCGCTTCGAGGC (H1A; SEQ ID NO: 16), CCCGCTCCTGCAGTATTAGTTCTTGC (H1B; SEQ ID NO: 17) and TTGCAGCTGGTGGTGGCGGCTGAGG (H1C; SEQ ID NO: 18). The antisense primers were TCCAATAGGGTCTTTGAGTCTGCCAC (H1D; SEQ ID NO: 19), CACTTTCAGCGCCTCCAGATCTGCC (H1E; SEQ ID NO: 20) and GCGAGGCAGGGGACAATAACAGATGG (H1F; SEQ ID NO: 21). The human RNase HII 3′cDNA derived from the EST sequence were amplified by polymerase chain reaction (PCR), using human liver Marathon ready cDNA (Clontech, Palo Alto, Calif.) as templates and H1A or H1B/AP1 (for first run PCR) as well as H1B or H1C/AP2 (for second run PCR) as primers. AP1 and AP2 are primers designed to hybridize to the Marathon ready cDNA linkers (linking cDNA insert to vector). The fragments were subjected to agarose gel electrophoresis and transferred to nitrocellulose membrane (Bio-Rad, Hercules Calif.) for confirmation by Southern blot, using a ³²P-labeled H1E probe (for 3′ RACE). The confirmed fragments were excised from the agarose gel and purified by gel extraction (Qiagen, Germany), then subcloned into a zero-blunt vector (Invitrogen, Carlsbad, Calif.) and subjected to DNA sequencing. The human RNase HII 5′ cDNA from the EST sequence was similarly amplified by 5′ RACE. The overlapping sequences were aligned and combined by the assembling programs of MacDNASIS v. 3.0 (Hitachi Software Engineering Co., America, Ltd.). The full length human RNase HII open reading frame nucleotide sequence obtained is provided herein as SEQ ID NO: 22. A culture containing this cDNA has been deposited with the American Type Culture Collection as ATCC deposit no. PTA-2897. Protein structure and analysis were performed by the program MacVector v6.0 (Oxford Molecular Group, UK). The 299-amino acid human RNase HII protein sequence encoded by the open reading frame is provided herein as SEQ ID NO: 1.

### Example 14

### Screening of the cDNA Library and Human RNase HII DNA Sequencing

A human liver cDNA lambda phage Uni-ZAP library (Stratagene, La Jolla, Calif.) was screened using the 3′ RACE products as specific probes. The positive cDNA clones were excised into pBluescript phagemid from lambda phage and subjected to DNA sequencing. Sequencing of the positive clones was performed with an automatic DNA sequencer by Retrogen Inc. (San Diego, Calif.).

### Example 15

### Northern Hybridization

Total RNA was isolated from different human cell lines (ATCC, Rockville, Md.) using the guanidine isothiocyanate method (21). Ten μg of total RNA was separated on a 1.2 % agarose/formaldehyde gel and transferred to Hybond-N+ (Amersham, Arlington Heights, Ill.) followed by fixing using UV crosslinker (Strategene, La Jolla, Calif.). The premade multiple tissue Northern Blot membranes were also purchased from Clontech (Palo Alto, Calif.). To detect RNase HII mRNA, hybridization was performed by using ³²P-labeled human RNase H II cDNA in Quik-Hyb buffer (Strategene, La Jolla, Calif.) at 68° C. for 2 hours. After hybridization, membranes were washed in a final stringency of 0.1×SSC/0.1% SDS at 60° C. for 30 minutes and subjected to auto-radiography.

RNase HII was detected in all human tissues examined (heart, brain, placenta, lung, liver, muscle, kidney and pancreas). RNase HII was also detected in all human cell lines tested (A549, Jurkat, NHDF, Sy5y, T24, MCF7, IMR32, HTB11, HUVEC, T47D, LnCAP, MRC5 and HL60) with the possible exception of NHDF for which presence or absence of a band was difficult to determine in this experiment. MCF7 cells appeared to have relatively high levels and HTB11 and HUVEC cells appeared to have relatively low levels compared to most cell lines.

### Example 16

### Expression and Purification of the Cloned Human RNase HII Protein

The cDNA fragment encoding the full human RNase HII protein sequence was amplified by PCR using 2 primers, one of which contains a restriction enzyme NdeI site adapter and six histidine (his-tag) codons and a 22-base pair protein N terminal coding sequence, the other contains an XhoI site and 24 bp protern C-terminal coding sequence including stop codon. The fragment was cloned into expression vector pET17b (Novagen, Madison, Wis.) and confirmed by DNA sequencing. The plasmid was transfected into E. coli BL21(DE3) (Novagen, Madison, Wis.). The bacteria were grown in LB medium at 37° C. and harvested when the OD₆₀₀, of the culture reached 0.8, in accordance with procedures described by Ausubel et al., (Current Protocols in Molecular Biology, Wiley and Sons, New York, N.Y., 1988). Cells were lysed in 8M urea solution and recombinant protein was partially purified with Ni-NTA agarose (Qiagen, Germany). Further purification was performed with C4 reverse phase chromatography (Beckman, System Gold, Fullerton, Calif.) with 0.1% TFA water and 0.1% TFA acetonitrile gradient of 0% to 80% in 40 minutes as described by Deutscher, M. P., (Guide to Protein Purification, Methods in Enzymology 182, Academic Press, New York, N.Y., 1990). The recombinant proteins and control samples were collected, lyophilized and subjected to 12% SDS-PAGE as described by Ausubel et al. (1988) (Current Protocols in Molecular Biology, Wiley and Sons, New York, N.Y.). The purified protein and control samples were resuspended in 6 M urea solution containing 20 mM Tris HCl, pH 7.4, 400 mM NaCl, 20% glycerol, 0.2 mM PMSF, 40 mM DTT, 10 μg/ml aprotinin and leupeptin, and refolded by dialysis with decreasing urea concentration from 6 M to 0.5 M as well as DTT concentration from 40 mM to 0.5 mM as described by Deutscher, M. P., (Guide to Protein Purification, Methods in Enzymology 182, Academic Press, New York, N.Y., 1990). The refolded proteins were concentrated (10 fold) by Centricon (Amicon, Danvers, Mass.) and subjected to an RNase H activity assay as described in the subsequent example.

### Example 17

### Human RNase H Activity Assay

A ³²P-end-labeled 17-mer RNA, GGGCGCCGTCGGTGTGG (SEQ ID NO: 23) described by Lima, W. F. and Crooke, S. T. (Biochemistry, 1997 36, 390-398), was gel-purified as described by Ausubel et al. (Current Protocols in Molecular Biology, Wiley and Sons, New York, N.Y., 1988) and annealed with a tenfold excess of its complementary 17-mer oligodeoxynucleotide. Annealing was done in 10 mM Tris HCl, pH 8.0, 10 mM MgCl, 50 mM KCl and 0.1 mM DTT to form one of two different substrates: single strand (S) RNA probe and full double strand (ds) RNA/DNA duplex. Each of these substrates was incubated with cloned and expressed RNase H protein (RNase HI or HII, isolated as described in the previous examples at 37° C. for 5 minutes to 60 minutes at the same conditions used in the annealing procedure and the reactions were terminated by adding EDTA in accordance with procedures described by Lima, W. F. and Crooke, S. T. (Biochemistry, 1997, 36, 390-398). The reaction mixtures were precipitated with TCA centrifugation and the supernatant was measured by liquid scintillation counting (Beckman LS6000IC, Fullerton, Calif.). An aliquot of the reaction mixture was also subjected to denaturing (8 M urea) acrylamide gel electrophoresis in accordance with procedures described by Lima, W. F. and Crooke, S. T. (Biochemistry, 1997, 36, 390-398) and Ausubel et al. (Current Protocols in Molecular Biology, Wiley and Sons, New York, N.Y., 1988). The gels were then analyzed and quantified using a Molecular Dynamics PhosphorImager.

Renatured recombinant human RNase HI displayed RNase H activity. Incubation of 10 ng purified renatured human RNase HI with RNA/DNA substrate for 2 hours resulted in cleavage of 40% of the substrate. The enzyme also cleaved RNA in an oligonucleotide/RNA duplex in which the oligonucleotide was a gapmer with a 5-deoxynucleotide gap, but at a much slower rate than the full RNA/DNA substrate. This is consistent with observations with E. coli RNase HI (Lima, W. F. and Crooke, S. T., Biochemistry, 1997, 36, 390-398). It was inactive against single-stranded RNA or double-stranded RNA substrates and was inhibited by Mn²⁺. The molecular weight (˜36 kDa) and inhibition by Mn²⁺ indicate that the cloned enzyme is highly homologous to E. coli RNase HI and has properties consistent with those assigned to Type 2 human RNase H.

The initial rates of RNase HI cleavage were determined for several duplex substrates studies simultaneously. The initial rate of cleavage for a phosphodiester 17mer DNA-RNA duplex was 1050±203 pmol liter⁻¹ min⁻¹ and the initial rate of cleavage of a phosphorothioate 17mer oligodeoxynucleotide-DNA duplex of the same sequence was approximately four-fold faster (4034±266 pmol liter⁻¹ min⁻¹). The initial rate for a 20-mer phosphodiester oligodeoxynucleotide-DNA duplex of unrelated sequence (targeted to hepatitis C virus core protein coding region) was 1015±264 pmol liter⁻¹ min⁻¹, equivalent to the 17mer ras sequence. However, a phosphorothioate 25mer oligodeoxynucleotide-DNA duplex, in which the 25mer oligonucleotide sequence was a ras sequence containing the 17mer ras sequence used above, was approximately 50% faster (1502±182 pmol liter⁻¹ min⁻¹).

Duplexes in which the antisense oligonucleotide was modified in the 2′ position were studied. Human RNase HI was unable to cleave substrates with 2′ modifications at the cleavage site of the antisense DNA strand or sense RNA 17mer strand, as shown in Table 4. All oligonucleotides are full phosphorothioates of SEQ ID NO: 6. 2′-methoxy (2′-O-methyl) modifications are shown in bold.

TABLE 4
Effects of 2′ substitution and deoxy-gap size on cleavage
rates by human RNase HI

| | Initial cleavage rate |
|---|---|
| Antisense DNA | (pmol liter⁻¹ min⁻¹) |
| CCACACCGACGGCGCCC | 4023 ± 266 |
| CCACACCGACGGCGCCC | 1081 ± 168 |
| CCACACCGACGGCGCCC | 605 ± 81 |
| CCACACCGACGGCGCCC | 330 ± 56 |
| CCACACCGACGGCGCCC | 0 |
| CCACACCGACGGCGCCC | 0 |
| CCACACCGACGGCGCCC | 0 |

A 20-mer duplex which had a 2′-propoxy (2′-O-propyl) modification at every nucleotide of the antisense phosphorothiate oligonucleotide was also tested similarly but, as with the full 2′-methoxy oligonucleotide, no cleavage was observed. A duplex in which the antisense oligonucleotide (of the same sequence) was 2′ deoxy at every position was cleaved (initial rate 3400 pmol liter⁻¹ min⁻¹).

The sites of RNA strand cleavage by human RNase HI were determined for both the full RNA/DNA substrate and the gapmer/RNA duplexes (in which the oligonucleotide gapmer had a 5-deoxynucleotide gap). In the full RNA/DNA duplex, the principal site of cleavage was near the middle of the substrate, with evidence of less prominent cleavage sites 3′ to the primary cleavage site. The primary cleavage site for the gapmer/RNA duplex was located across the nucleotide adjacent to the junction of the 2′ methoxy wing and oligodeoxy nucleotide gap nearest the 3′ end of the RNA. Thus, the human RNase HI enzyme resulted in a major cleavage site in the center of the RNA/DNA substrate and less prominent cleavages to the 3′ side of the major cleavage site. The shift of its major cleavage site to the nucleotide in apposition to the DNA 2′ methoxy junction of the 2′ methoxy wing at the 5′ end of the chimeric oligonucleotide is consistent with the observations for E. coli RNase HI (Crooke et al. (1995) Biochem. J. 312, 599-608; Lima, W. F. and Crooke, S. T. (1997) Biochemistry 36, 390-398). The fact that the enzyme cleaves at a single site in a 5-deoxy gap duplex indicates that the enzyme has a catalytic region of similar dimensions to that of E. coli RNase HI.

In this assay, cloned and expressed human RNase HII also demonstrated cleavage of the substrate RNA/DNA duplex. Cleavage was detectable after 60 minutes.

### Example 18

### Cleavage of 2′-methoxyethoxy Chimeric Oligonucleotides by Human RNase H

The following 2′-methoxyethoxy (2′-MOE) chimeric oligonucleotides (nucleotides with 2′-MOE modification are in bold) were tested as in the above example, using recombinant human RNase HI cloned and expressed as in the above examples. Each antisense oligonucleotide was tested against a length-matched sense oligonucleotide.

TABLE 5
Cleavage of 2′-MOE chimeric oligonucleotides by human RNase
HI

| ISIS # | Target | Sequence | SEQ ID NO: |
|---|---|---|---|
| 111629 | human | AGCTTCTTTGCACATGTAAA | 24 |
| | MDM2 | | |
| 22023 | murine | TCCAGCACTTTCTTTTCCGG | 25 |
| | Fas | | |
| 15493 | murine | CCGGTACCCCAGGTTCTTCA | 26 |
| | A-raf | | |

All 2′-MOE oligonucleotides tested were shown to elicit cleavage of their complementary target sequence by human RNase HI.

What is claimed is:

1. A method of promoting inhibition of expression of a selected protein by an antisense oligonucleotide targeted to an RNA encoding the selected protein comprising:
(a) providing an antisense oligonucleotide targeted to an RNA encoding a selected protein whose expression is to be inhibited, wherein said oligonucleotide is a chimeric oligonucleotide having a modification at the 2′ position of at least one sugar moiety;
(b) allowing said oligonucleotide and said RNA to hybridize to form an oligonucleotide-RNA duplex; and
(c) contacting said oligonucleotide-RNA duplex with a mammalian RNase H polypeptide, under conditions in which cleavage of the RNA strand of the oligonucleotide-RNA duplex occurs, whereby inhibition of expression of the selected protein is promoted.

2. The method of claim 1 wherein the mammalian RNase H polypeptide is a human RNase H polypeptide.

3. The method of claim 1 wherein the mammalian RNase H polypeptide is an RNase HI polypeptide.

4. The method of claim 1 wherein the mammalian RNase H polypeptide is an RNase HII polypeptide.

5. The method of claim 1 wherein the mammalian RNase H polypeptide is present in enriched amounts.

6. The method of claim 5 wherein the mammalian RNase H polypeptide present in enriched amounts is overexpressed or exogenously added.

7. The method of claim 1 wherein the mammalian RNase H polypeptide is an isolated, purified mammalian RNase H.

8. A method of eliciting cleavage of a selected cellular RNA target comprising:
(a) providing an antisense oligonucleotide targeted to a selected cellular RNA target to be cleaved, wherein said oligonucleotide is a chimeric oligonucleotide having a modification at the 2′ position of at least one sugar moiety;
(b) allowing said oligonucleotide and said RNA to hybridize to form an oligonucleotide-RNA duplex; and
(c) contacting said oligonucleotide-RNA duplex with a mammalian RNase H polypeptide, under conditions in which cleavage of the RNA strand of the oligonucleotide-RNA duplex occurs,whereby cleavage of the cellular RNA target is elicited.

9. The method of claim 8 wherein the mammalian RNase H polypeptide is a human RNase H polypeptide.

10. The method of claim 8 wherein the mammalian RNase H polypeptide is an RNase HI polypeptide.

11. The method of claim 8 wherein the mammalian RNase H polypeptide is an RNase HII polypeptide.

12. The method of claim 8 wherein the mammalian RNase H polypeptide is present in enriched amounts.

13. The method of claim 12 wherein the mammalian RNase H polypeptide present in enriched amounts overexpressed or exogenously added.

14. The method of claim 8 wherein the mammalian RNase H polypeptide is an isolated, purified mammalian RNase H.

15. A method of making an antisense oligonucleotide which elicits cleavage of its complementary target RNA by a mammalian RNase H polypeptide comprising synthesizing a chimeric oligonucleotide which has a modification at the 2′ position of at least one sugar moiety, and which is targeted to a selected RNA, wherein said oligonucleotide, when hybridized to the selected RNA target to form a duplex, will bind a mammalian RNase H polypeptide which thereby cleaves the RNA strand of the duplex.

16. The method of claim 15 wherein the mammalian RNase H polypeptide is a human RNase H polypeptide.

17. The method of claim 15 wherein the mammalian RNase H polypeptide is an RNase HI polypeptide.

18. The method of claim 15 wherein the mammalian RNase H polypeptide is an RNase HII polypeptide.
